(19) United States
(12) Patent Application Publication
(10) Pub. No.: US 2024/0286019 A1
(43) Pub. Date: Aug. 29, 2024

(54) MODULAR INTERACTIVE EXERCISE AND GAMING SYSTEMS AND METHODS

(71) Applicant: Hykso Inc., Costa Mesa, CA (US)

(72) Inventor: Alexandre Marcotte St-Miche, Costa Mesa, CA (US)

(73) Assignee: Hykso Inc., Costa Mesa, CA (US)

(21) Appl. No.: 18/115,419

(22) Filed: Feb. 28, 2023

(51) Int. Cl. A63B 71/06; A63B 24/00

(52) U.S. Cl. CPC A63B 71/0622; A63B 24/0062; A63B 24/0075; A63B 2209/08; A63B 2220/44; A63B 2220/836; A63B 2225/50; A63B 2230/06

(57) ABSTRACT

Described herein are user-friendly systems and methods that extend the functionality of interactive exercise and gaming systems comprising motion trackers. In certain embodiments, this is accomplished by providing a tracker module that mechanically and electrically couples to a console and, once authenticated, unlocks one or more tracker, exercise, or gaming features to enhance user motivation.

### BACKGROUND

### A. Technical Field

The present disclosure relates generally to interactive training equipment. More particularly, the present disclosure relates to user-friendly, integrated training and gaming equipment that utilizes the computational processing resources of a console to motivate users to engage in physical activities, such as martial arts-type physical exercises.

### B. Background

Interactive physical training equipment for home use enjoys increased popularity with fitness enthusiasts. Increased ease of customization, including gaming options provided by sensors and auxiliary devices allow for dynamic individualized workout. A user may engage in a large number of physical activities that oftentimes involve physical contact with a target with upper and lower limbs, such as a punching bag or kicking pad in boxing, martial arts, and similar activities. Several existing devices monitor a user's movements and provide at least some form of feedback. Yet, many such devices suffer from various drawbacks, including being bulky, heavy, and limited in functionality such as the inability to add to an existing system a sufficiently large number of trackers to accommodate a relatively large group of users. In addition to limited connectivity options in existing devices, each tracker that communicates over a Bluetooth connection must undergo a cumbersome process of being individually paired with a device such as a smartphone or tablet, further negatively impacting the user experience. Such limited functionality tends to cause users to relatively quickly loose interest in physical activities and exercises provided even by physical training equipment having gaming features and.

Accordingly, what is needed are user-friendly exercise and gaming systems and methods that enhance functionality, such as group workout routines that aid in providing and maintaining user motivation.

### BRIEF DESCRIPTION OF THE DRAWINGS

References will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments. Items in the figures are not to scale.

Figure (“FIG.”) 1 is a general illustration of a portable interactive exercise and gaming platform according to various embodiments of the present disclosure.

FIG. 2 is a perspective view of an exemplary implementation of the console shown in FIG. 1.

FIG. 3 is a block diagram illustrating an exemplary tracker according to various embodiments of the present disclosure.

FIG. 4 is a block diagram illustrating an exemplary console according to various embodiments of the present disclosure.

FIG. 5 is a flowchart of an illustrative process for using an interactive exercise and gaming platform according to various embodiments of the present disclosure.

### DETAILED DESCRIPTION OF EMBODIMENTS

In the following description, for purposes of explanation, specific details are set forth in order to provide an understanding of the invention. It will be apparent, however, to one skilled in the art that the invention can be practiced without these details. Furthermore, one skilled in the art will recognize that embodiments of the present invention, described below, may be implemented in a variety of ways, such as a process, an apparatus, a system, a device, or a method on a tangible computer-readable medium.

Components, or modules, shown in diagrams are illustrative of exemplary embodiments of the invention and are meant to avoid obscuring the invention. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components. Components may be implemented in software, hardware, or a combination thereof.

Furthermore, connections between components or systems within the figures are not intended to be limited to direct connections. Rather, data between these components may be modified, re-formatted, or otherwise changed by intermediary components. Also, additional or fewer connections may be used. It shall also be noted that the terms “coupled,” “connected,” or “communicatively coupled” shall be understood to include direct connections, indirect connections through one or more intermediary devices, and wireless connections.

Reference in the specification to “one embodiment,” “preferred embodiment,” “an embodiment,” or “embodiments” means that a particular feature, structure, characteristic, or function described in connection with the embodiment is included in at least one embodiment of the invention and may be in more than one embodiment. Also, the appearances of the above-noted phrases in various places in the specification are not necessarily all referring to the same embodiment or embodiments.

The use of certain terms in various places in the specification is for illustration and should not be construed as limiting. A service, function, or resource is not limited to a single service, function, or resource; usage of these terms may refer to a grouping of related services, functions, or resources, which may be distributed or aggregated.

The terms “include,” “including,” “comprise,” and “comprising” shall be understood to be open terms and any lists the follow are examples and not meant to be limited to the listed items. Any headings used herein are for organizational purposes only and shall not be used to limit the scope of the description or the claims. Each reference mentioned in this patent document is incorporate by reference herein in its entirety.

As mentioned in the background, existing tracking devices containing sensors have limited connectivity options. Due to their limited processing power, memory, etc., these devices are unable to perform sophisticated calculations or algorithms locally and, thus, must typically rely on a smartphone for connectivity. It is highly desirable to reduce or eliminate sensors' dependency on the smartphone for connectivity.

FIG. 1 is a general illustration of a portable interactive exercise and gaming platform according to various embodiments of the present disclosure. In embodiments, exercise and gaming platform 100 comprises console 102, trackers 106-109, display device 110, tracker module 140. It is understood that any connection herein between displayed components may represent bidirectional communication paths. It is understood that console 102 may comprise any number of communication interfaces (not shown in FIG. 1), e.g., to support multiple users. In embodiments, console 102 may further comprise any number of charging slots 104 that corresponds to a number of trackers that can be received, may serve both as a charging dock and a gateway for trackers 106-109. Similarly, tracker module 140 may comprise one or more charging slots 142 designed to receive and charge one or more trackers (not shown).

In operation, a user engaged in a physical activity, including exercising or gaming may attach one or more trackers to upper and lower limbs. For example, tracker 106 may be attached to a user's wrist, and tracker 107 may be attached to the user's ankle, such that movements such as punching and kicking a target can be detected and monitored in the form of motion data, e.g., to provide real-time feedback to improve reaction times. Another set of trackers (e.g., 108, 109) may be attached to a second user's wrist and ankle during a workout program, or simply to facilitate competition between participants in a competitive setting or game. However, this is not intended as a limitation on the scope of the present disclosure. As discussed in greater detail below, in embodiments, trackers 106-109 may be attached to equipment that involves activities that may be performed with no physical contact at all, such as jump roping, weight lifting, and so on.

In embodiments, trackers 106-109 may wirelessly communicate with console 102, primarily to exchange data, such as motion data. Trackers 106-109 may be implemented as any type of motion sensor or device. As an example, trackers 106-109 may comprise a three-axis accelerometer that once activated is capable of detecting acceleration in three dimensions, e.g., by measuring and/or calculating data at a certain sampling rate. In motion sensor applications, trackers 106-109 may further comprise a gyroscope that capable of detecting an orientation of a tracker at any moment in time.

Each of trackers 106-109 may communicate the results of measurements to console 102, e.g., at predetermined time intervals. In embodiments, the communication by trackers 106-109 may be daisy-chained at console 102, which may independently monitor each of tracker 106-109.

In embodiments, in addition to console 102 being able to act as a local server to which any number devices 110 may connect, e.g., in a LAN configuration via a Bluetooth connection, console 102 may establish a connection with remote server 130 to communicate, e.g., raw data that has been gathered by trackers 106-109 to server 130. As discussed in greater detail below, server, such as server 130, may be used to process and analyze the received data, e.g., to analyze and modify an exercise program to improve user experience.

In embodiments, console 102 may further communicate over any known interface with display device 110, which may be implemented for example in a smartphone or television to allow a user to control certain functions of console 102. Communication between console 102 and display device 110 may be established using any known communication method and communication protocol known in the art. For example, display device 110 may use a Bluetooth connection to automatically detect console 102. Alternatively, console 102 may comprise a HDMI port that may connect with a counterpart HDMI port on a television set or any other HDMI-enabled device 110. Furthermore, it is understood that console 102 may communicate with any number of peripheral devices (not shown), such as a headphone, speaker, or any other electronic device. Moreover, console 102 may communicate with the internet or other network, including server 130, e.g., to download applications and/or data, such as audio or video data, from server 130 that may be merged and analyzed together with data that console 102 monitors regarding trackers 106-109.

In embodiments, once a user utilizes platform 100 to engage in various interactive sessions of a program that may be presented to the user on display device 110, e.g., a gaming session or instruction-based (e.g., trainer-led) exercise program comprising a type of physical activity for one or more participants, console 102 may execute steps of an algorithm that may have been provided by server 130 and communicate with display device 110 to display instructions and other information to the user. Display device 110 may comprise a user interface capable of receiving information input by a user, such as height and weight data, which the user may enter. Server 130 may maintain and control any number of programs and program features that correspond to a sessions that may be activated on demand. In embodiments, console 102 may receive and store any number of pre-programmed and user-selectable exercise programs or games that a user may directly or indirectly activate. It is noted that games such as reaction tests and various exercise-related games may or may not have a major physical component.

In embodiments, console 102 may receive motion-related data captured by trackers 106-109 that correspond to movements performed by the user, such as a punching or kicking motion, which trackers 106-109 may monitor and communicate to console 102, e.g., as measured raw data or pre-processed motion-related data. Console 102 may process some or all of the data to determine, e.g., a count or number of punches that satisfy a criterion related to a measurable property such as timing, speed, etc., and derive any number of other metrics, such as reaction time, accuracy, force, and the like, which may be presented to the user on display device 110 in any suitable format. For example, console 102 may calculate and present to users a score, e.g., at predetermined intervals or after reaching certain steps of an exercise session. Scores related to a performance measure, a measure of competitiveness among two or more users that engage in a competitive group activity, and the like may serve as a motivational tool to keep users engaged in physical activities in the short-term and long-term.

In embodiments, display device 110 may provide any type of personalized feedback related to an exercise or a game for any number of users. As an example, display device 110 may provide visible and/or audible feedback to a user based on the information trackers 106-109 provide to console 102. In embodiments, display device 110 may display an animated avatar for various purposes in a session. The avatar may represent the user, an instructor providing instructions for the user to follow, or a partner or opponent. Gaming sessions or exercise sessions may have any number of local (e.g., sharing console 102) and remote participants (e.g., using a different console and/or exercise system. Gaming embodiments of system 100, may use an avatar that is displayed on display device 110 to indicate, for example, whether a movement tracked by one or more of trackers 106-109 has been correctly performed.

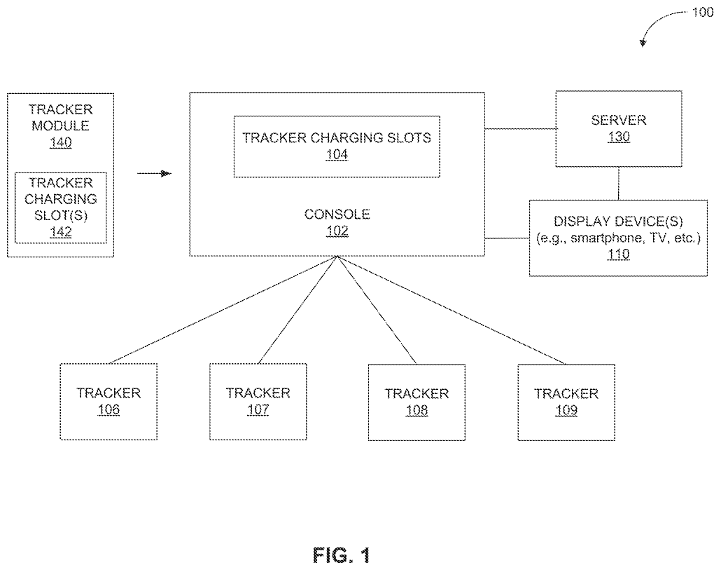

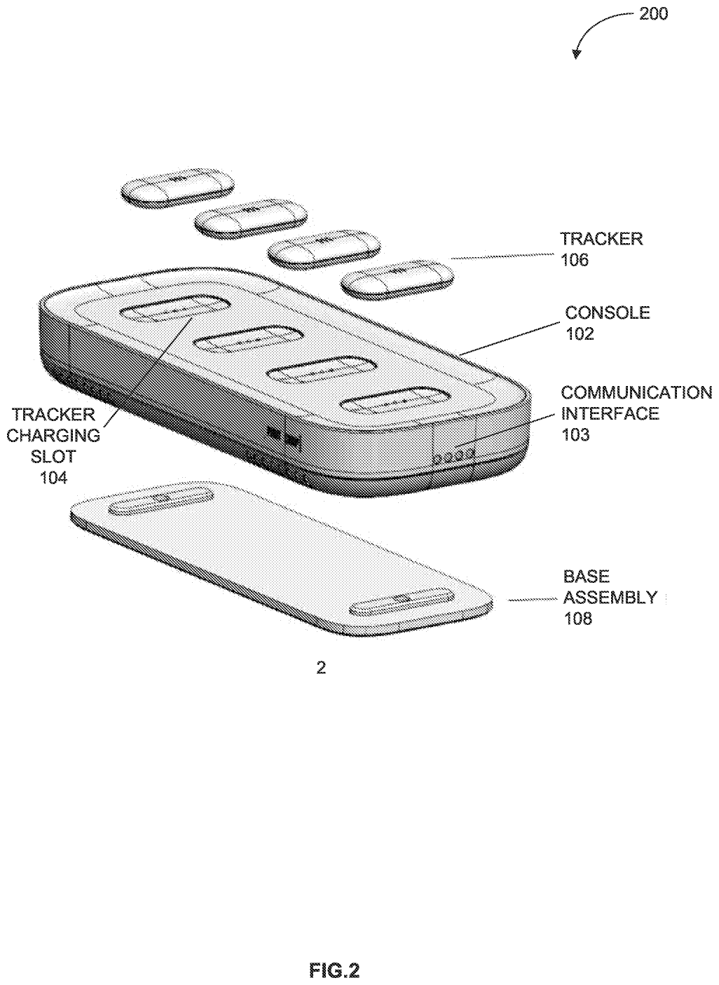

FIG. 2 is a perspective view of an exemplary implementation of the console shown in FIG. 1. For clarity, components similar to those shown in FIG. 1 are labeled in the same manner. For purposes of brevity, a description or their function is not repeated here. As depicted, console 102 may comprise four charging slots (e.g., 104), communication interface 103, and detachable base plate 108. In embodiments, tracker 106 may be hermetically sealed, e.g., via an O-ring such as to make the housing of tracker 106 waterproof and prevent accidental water damage.

In various applications, tracker 106 may comprise sensors or probes (not shown), such as electrical contacts that in operation may be attached to the user's body or skin to gather physiological data associated with a user's body or information related to certain physical characteristics. Exemplary sensor data comprises heart rate data, skin conductivity data (e.g., using a galvanic skin response method to determine a sweat rate therefrom), blood pressure data, and caloric expenditure data just to name a few. Physical parameters may comprise previously mentioned acceleration data that is representative of movements of various body parts. It is understood that sensors embedded in tracker 106 may gather data continuously or at certain periodical, random, or user-defined intervals.

As depicted, exemplary console 102 may be implemented in a portable housing that may be removably mounted on detachable base plate 108. Base plate 108 may be permanently affixed to a surface, such as a wall, e.g., in a garage or other suitable location. Console 102 in FIG. 2 comprises four tracker charging slots (e.g., slot 104) recesses dimensioned large enough to receive tracker 106 and serve as a mechanism for removably attaching tracker 106 to console 102. In embodiments, tracker 106 may comprise magnetic material that allows tracker 106 to magnetically and/or electrically connect to charging slot 104, e.g., to enable tracker 106 to be electrically charged, e.g., via a number of electrical contacts, such as charging pins. Magnetic coupling between tracker 106 and charging slot 104 enables charging slot 104 exert a mechanical force that acts on tracker 106 and pulls together tracker 106 and charging slot 104 such as to establish a removable mechanical connection with each other. In embodiments, the magnetic material in tracker 106 may be implemented as a magnet made of any material having intrinsic magnetic properties, such as a neodymium iron boron permanent magnet that may be permanently affixed to tracker 106 and may have any geometrical shape. Conversely, magnetic material in charging slot 104 may be implemented as a permanent magnet, a metal core, or a magnetically conductive surface layer or coating. The magnetic material, or more accurately the magnetic forces resulting from the magnetic circuit created by the magnetic material, allow charging slot 104 to magnetically couple to tracker 106 to establish the removable mechanical connection.

In embodiments, the magnetic properties of tracker 106 allow tracker 106 to magnetically, and thus mechanically, couple to certain exercise equipment that contains ferromagnetic material such as iron, which may be found in dumb bells, steel swords, etc., that makes such exercise equipment inherently suitable for direct attachment with magnetic tracker 106. In embodiments, tracker 106 may be attached to exercise equipment that does not have inherent non-magnetic properties, such as jump ropes, e.g., by embedding tracker 106 into a slot or cavity within the exercise equipment. Alternatively, tracker 106 may be removably attached to the user's clothing. Certain clothing known as “wearables” may be specifically designed to have any number of locations at which tracker 106 may be attached, e.g., depending on a specific sport or exercise program.

In embodiments, once tracker charging slot 104 receives tracker 106, console 102 may electrically charge a rechargeable battery located within the housing of tracker 106, e.g., by any known inductive charging method known in the art. In embodiments, charging may occur independently of the orientation of tracker 106 within charging slot 104. As a result, tracker 106 may advantageously be charged in any arbitrary orientation, thus, avoiding negatively affecting user experience as users would otherwise have to figure out a proper orientation for each tracker 106, or undergo a cumbersome process to troubleshoot the equipment in case of a misalignment.

It is noted that console 102 illustrated in FIG. 2 is not limited to the constructional detail shown there or described in the accompanying text. In embodiments, console 102 may be made extendible and designed in a modular fashion to receive one or more tracker modules 140. Each tracker module 140 may comprise any number of trackers, sensors, or other devices, such as a heart rate monitor, to enhance user experience. Console 102 may comprise one or more receptacles and/or extension ports that may receive tracker modules 140 to console 102. Each tracker module 140, which may be viewed as a secondary console that comprises a charging unit and tracker chagrin slot 142, may be mechanically coupled to console 102, e.g., via a magnetic interface. Further, each tracker module 140, or accessory to the primary console, may comprise a counterpart interface that electrically couples to interface 103 to be receive electrical power from by console 102, e.g., via an I²C connection, or any other method known in the art.

Advantageously, such a modular design allows console 102 to detect the presence of additional tracker modules (e.g., module 140) and allows any number of additional trackers to stream data to console 102. For example, tracker module 140 with a rechargeable tracker that comprises a heart rate sensor may, upon electrically and mechanically coupling to console 102, be powered by console 102. Further, the tracker associated with tracker module 140 may stream heart rate sensor data to console 102, e.g., via a Bluetooth connection.

In embodiments, a display device (not shown in FIG. 2) may display an identifier (e.g., a serial number) over a user interface to each tracker module 140 that has been added to console 102 to indicate the type of device that is connected. In embodiments, once console 102 detects that module/device 140 has been connected to console 102, console 102 may generate an acoustic signal to indicate to a user that the connection has been established. Console 102 may start receiving and reading data, e.g., data advertised by tracker 106 and comprising the identifier that identifies tracker 106 prior to console 102 permitting tracker 106 to stream data to console 102.

In embodiments, once module 140 is connected to console 102 and has undergone a start-up procedure, module 140 may take control over the I²C bus and communicate its presence to console 102. Console 102 may use the identifier to unlock in an app some content or functionality associated with module 140. It is understood that console 102 may log any communication and that each module 140 may comprise its own processing device.

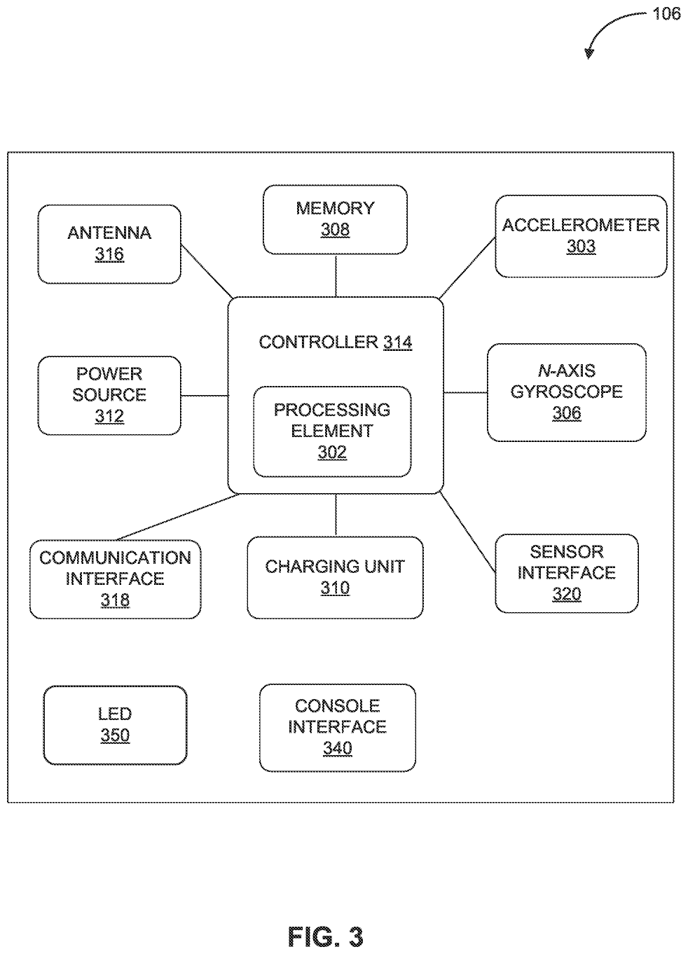

FIG. 3 is a block diagram illustrating an exemplary tracker according to various embodiments of the present disclosure. As depicted, tracker 106 may comprise controller 314, which may comprise processing element 302; antenna 316; memory 308; power source 312, e.g., a rechargeable battery; wired or wireless communication interface 318; motion sensors, such as accelerometer 303 and gyroscope 306; any number and type of other trackable devices and sensor interfaces 320; console interface 340, e.g., a magnetic connection interface; and LED 350.

In embodiments, tracker 106 may use communication interface 318 to, e.g., wirelessly communicate via any type of wireless communication (e.g., Bluetooth) with a console (not shown in FIG. 3) without requiring user input, e.g., after automatically exchanging device credentials. In embodiments, tracker 106 may use communication interface 318 and, e.g., a proprietary communication protocol to communicate a set of tracker parameters to communicate raw data to the console during regular operation.

In embodiments, after establishing communication with the console, e.g., as part of a calibration routine, a user may be prompted to execute a number of initial movements or exercises, which tracker 106 or the console may use to identify the location of each tracker 106, for example to determine whether tracker 106 has been attached to the user's left or right wrist or ankle.

In regular operation, tracker 106 pre-process and store some or all of gathered sensor data, such as accelerometer and gyroscope data, prior to passing such data to interface 318 for communication with the console. The console may use the data, e.g., to determine that the data represents a strike by the user's left hand. In embodiments, data gathered by tracker 106 may serve as user feedback, e.g., to evaluate compliance with a user exercise, or provide feedback to the user related to the exercise, such as success rates, etc. Such feedback equally applies to gaming features, competitions, and other activities and enhances the user's engagement with the equipment. To further increase the level of user engagement, feedback may comprise information related to one or more training/gaming partners.

In embodiments, e.g., in a learning or lesson mode, tracker 106 may be used for pose tracking and form correction, such as reminding the user to keep hands at a certain height, e.g., relative to the head when performing punching exercises. In embodiments, such information may further be used as parameter for monitoring and evaluating the user's physical condition, e.g., to make a determination regarding the user's level of exhaustion, to adjust an exercise routine, to increase the efficiency of an exercise routine, and the like. In embodiments, one or more external devices, e.g., a smartphone camera may be leveraged to enable sophisticated tracking of movements of individual body parts and associated feedback to achieve enhanced form correction.

In embodiments, to reduce overall power consumption, controller 314 may comprise circuitry that enables tracker 106 to switch from a data gathering mode during periods of relatively low activity in which tracker 106 consumes relatively low power to activities involving bursts of relatively high-power communication. In addition, controller 314 may monitor and control, high-power circuit components and sub-components, such as gyroscope 306, to control power consumption, for example, by facilitating activation and deactivation operations of internal and external communication of data. In embodiments, this may comprise causing one or more circuit components to strategically enter and exit a sleep mode to, advantageously, reduce power consumption and extend the availability of power source 312 and, thus, the availability of the overall system.

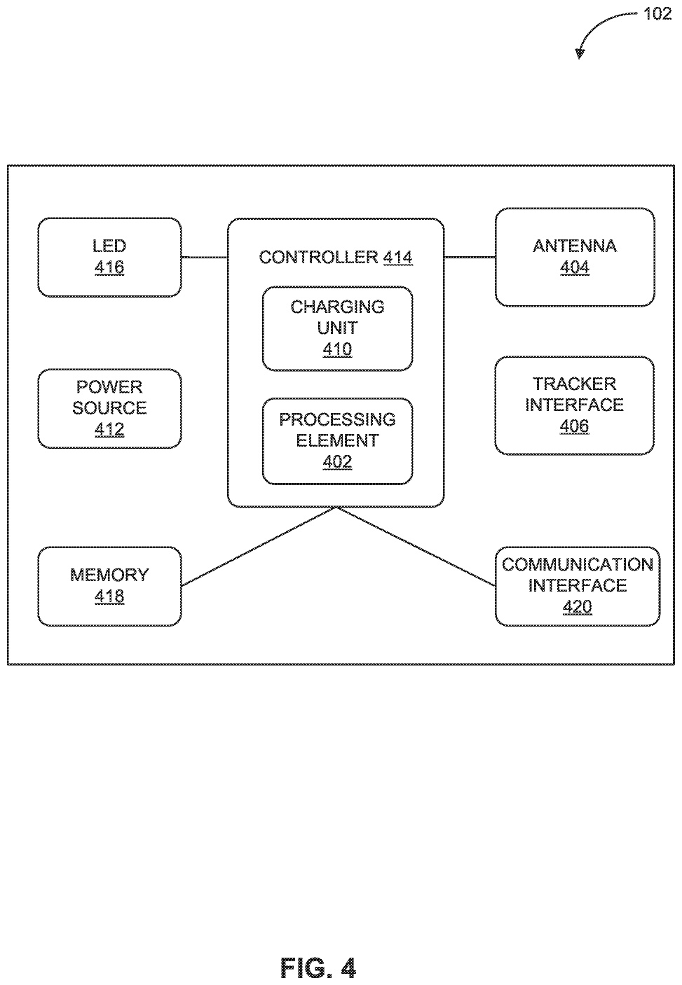

FIG. 4 is a block diagram illustrating an exemplary console according to various embodiments of the present disclosure. In a manner similar to FIG. 3, console 102 may comprise controller 414, which may comprise processing element 402 and charging element 410. Console 102 in FIG. 4 may further comprise antenna 404; power source 412, e.g., a rechargeable battery; wired or wireless communication interface 420, e.g., one or more Ethernet ports; tracker interface 406, e.g., a memory device 418; and a set of LEDs 416 or equivalent circuitry to communicate audible and/or visual alerts and other information, e.g., in different colors or on and off sequences. It is understood that, like processing element 302 in FIG. 3, processing element 402 in FIG. 4 may be implemented as a standalone processor, such as a microcontroller, or as a distributed processing units.

Memory device 418 may store a number of programs that may comprise an exercise routine or a game that console 102 may communicate to a user interface, e.g., on display device 110 shown in FIG. 1. An exemplary program may display data related to measured or otherwise detected motion that may have been captured by trackers 106-109 in FIG. 1 that have been placed on the user's right and left wrist or gloves during an exercise session. Instructions regarding an exercise routine, which may involve a sequence of punches that the user throws with alternating hands to strike a target such as a punching bag, may be displayed on display device 110, e.g., as an instructional animation. Motion data detected by sensors such as an accelerometer in tracker 106 may also be displayed, e.g., after being evaluated by being compared to expected data and generating a score indicating accuracy, timing, or any other metric.

In embodiments, Bluetooth hardware may be implemented as a dedicated integrated circuit that communicates with trackers 106-109 and may, e.g., in a setup phase, allow one or more of trackers 106-109 to wirelessly communicate and pair with a smartphone or other device 110, e.g., by exchanging user, Wi-Fi, and/or device credentials, without requiring the user to input information for each individual tracker. In embodiments, after establishing communication with trackers 106-109, console 102 may define or adjust tracker parameters. It is understood that console 102 may wirelessly communicate over a set of dedicated channels with trackers 106-109 that may, e.g., in a pseudo-parallel communication method, communicate raw data to console 102.

In embodiments, console 102 may communicate the data received from tracker 106-109, in any format, with and without further processing, to a remote server that may analyze the data. In embodiments, console 102 may correlate tracker data with timestamp data, e.g., to identify certain exercises, such as jumping jacks, sit-ups, etc., that a user wearing a set of trackers performs. The timestamp data, or data derived therefrom, may, thus, advantageously be used to label a set of tracker data. Server 130 may then use the labeled data, e.g., in a machine learning process, to improve overall data analysis, user activities, such as exercise programs and games to, ultimately, enhance user experience.

In embodiments, an activity such as a game that need not directly be related to a particular exercise may be unlocked and made available, e.g., in response to a monitored exercise indicating that the user's performance has satisfied a predetermined condition.

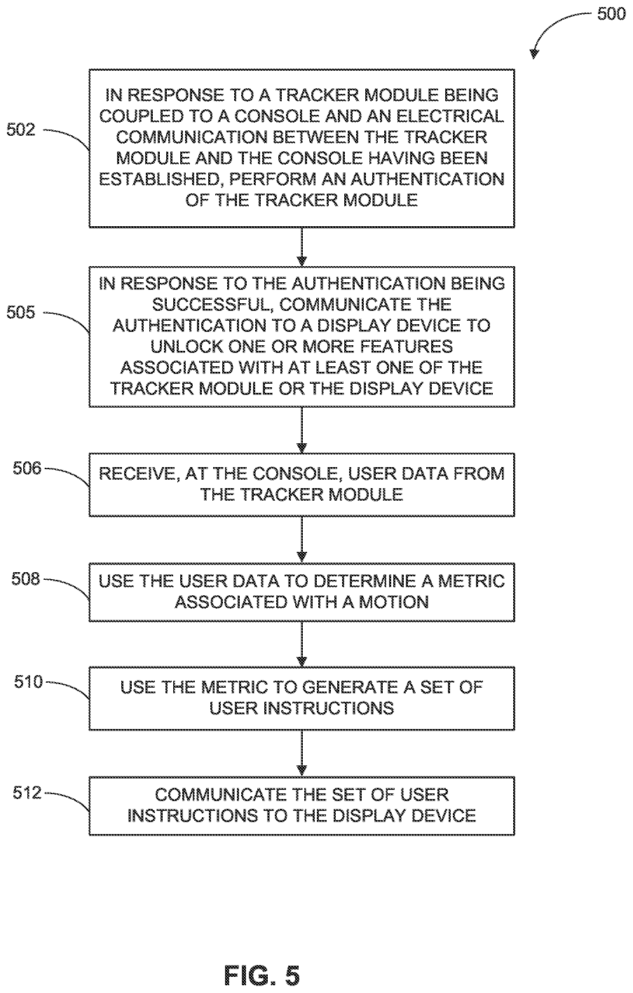

FIG. 5 is a flowchart of an illustrative process for using an interactive exercise and gaming platform according to various embodiments of the present disclosure. In embodiments, The process for using an interactive exercise and gaming platform may start at step 502 when, in response to a user coupling a tracker module to a console such as to establish an electrical communication between the tracker module and the console, the console may perform an authentication of the tracker module, e.g., by using a unique identifier.

At step 704, in response to the console deeming the authentication as successful, the console may communicate the result to a display device, such as a smartphone or other user interface, e.g., to unlock one or more features associated with the tracker module or the display device. At step 706, in the console may receive user data from the tracker module. At step 708, the user data may be used to determine a metric that is associated with a motion, e.g., a motion in an exercise routine. At step 710, the console may then use the metric, e.g., to generate a set of user instructions that, at step 712, may be communicated to the user interface.

One skilled in the art shall recognize that: (1) certain steps may optionally be performed; (2) steps may not be limited to the specific order set forth herein; (3) certain steps may be performed in different orders; and (4) certain steps may be done concurrently.

In embodiments, aspects of the present patent document may be directed to, may include, or may be implemented on one or more information handling systems/computing systems. A computing system may include any instrumentality or aggregate of instrumentalities operable to compute, calculate, determine, classify, process, transmit, receive, retrieve, originate, route, switch, store, display, communicate, manifest, detect, record, reproduce, handle, or utilize any form of information, intelligence, or data. For example, a computing system may be or may include a personal computer (e.g., laptop), tablet computer, phablet, personal digital assistant (PDA), smart phone, smart watch, smart package, server (e.g., blade server or rack server), a network storage device, camera, or any other suitable device and may vary in size, shape, performance, functionality, and price. The computing system may include random access memory (RAM), one or more processing resources such as a central processing unit (CPU) or hardware or software control logic, ROM, and/or other types of memory. Additional components of the computing system may include one or more disk drives, one or more network ports for communicating with external devices as well as various input and output (I/O) devices, such as a keyboard, a mouse, touchscreen and/or a video display. The computing system may also include one or more buses operable to transmit communications between the various hardware components.

Aspects of the present invention may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present invention may further relate to computer products with a non-transitory, tangible computer-readable medium that have computer code thereon for performing various computer-implemented operations. The media and computer code may be those specially designed and constructed for the purposes of the present invention, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present invention may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present invention. It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

What is claimed is:

1. A method for extending the functionality of an interactive exercise platform, the method comprising:
in response to a tracker module being coupled to a console and an electrical communication between the tracker module and the console having been established, performing an authentication of the tracker module; and
in response to the authentication being successful, communicating the authentication to a display device to unlock one or more features associated with at least one of the tracker module or the display device.

2. The method of claim 1, further comprising:
receiving, at the console, user-related data from the tracker module;
using the user-related data to determine a metric associated with a motion;
using the metric to generate a set of user instructions; and
communicating the set of user instructions to the display device.

3. The method of claim 1, wherein the tracker module comprises a heart rate sensor.

4. The method of claim 2, further comprising comparing the user-related data to existing data to determine whether the metric satisfies a condition.

5. The method of claim 4, wherein the metric is associated with at least one of acceleration information or orientation information associated with at least one user.

6. The method of claim 2, further comprising communicating the user-related data to a server to update one or more instructions in the set of user instructions and communicate the one or more instructions to the console.

7. The method of claim 1, wherein the console comprises a set of recesses that each, in response to a tracker being magnetically attached to the recess, inductively charges the tracker.

8. The method of claim 7, wherein the console comprises a ferromagnetic material.

9. The method of claim 8, wherein the ferromagnetic material is least one of a permanent magnet or a magnetically conductive surface layer to magnetically couple to the tracker.

10. A console for interactive exercise and gaming, the console comprising:
a set of tracker charging slots to receive a set of trackers;
a first communication interface to communicate with the set of trackers;
a receptacle to receive a tracker module; and
a second communication interface to communicate with a display device.
a processor; and
a non-transitory computer-readable medium comprising instructions that, when executed by the processor, cause steps to be performed, the steps comprising:
in response to the tracker module being coupled to the receptacle, establishing an electrical communication with the tracker module;
performing an authentication of the tracker module;
in response to the authentication being successful, communicating the authentication to the display device to unlock one or more features associated with at least one of the tracker module or the display device;
in response to receiving user-related data from the tracker module, determining a metric associated with a motion to generate a set of user instructions; and
communicating the set of user instructions to the display device.

11. The console of claim 10, wherein the tracker module comprises a heart rate sensor.

12. The console of claim 10, wherein the steps further comprise comparing the user-related data to existing data to determine whether the metric satisfies a condition.

13. The console of claim 12, wherein the metric is associated with at least one of acceleration information or orientation information associated with at least one user.

14. The console of claim 10, wherein the steps further comprise communicating the user-related data to a server to update one or more instructions in the set of user instructions and communicate the one or more instructions to the console.

15. The console of claim 10, further comprising a set of recesses that each, in response to a tracker being magnetically attached to the recess, inductively charges the tracker.

16. The console of claim 15, further comprising a ferromagnetic material that is least one of a permanent magnet or a magnetically conductive surface layer to magnetically couple to the tracker.

17. An interactive exercise and gaming system comprising:
a server; a
a display device.
a set of trackers comprising a set of detectors that gather user-related data;
a console comprising:
a set of tracker charging slots to receive a first set of trackers;
a receptacle to receive a tracker module to receive a second set of trackers;
a second communication interface to communicate with the display device.
a processor; and
a non-transitory computer-readable medium comprising instructions that, when executed by the processor, cause steps to be performed, the steps comprising:
in response to the tracker module being coupled to the receptacle, establishing an electrical communication with the tracker module;
communicating with the display device to unlock one or more features associated with at least one of the tracker module or the display device;
in response to receiving the user-related data, determining a metric associated with a motion to generate a set of user instructions; and
communicating the set of user instructions to the display device.

18. The system of claim 17, wherein the steps further comprise comparing the user-related data to existing data to determine whether the metric satisfies a condition, the metric being associated with at least one of acceleration information or orientation information associated with at least one user.

19. The system of claim 17, wherein the steps further comprise communicating the user-related data to the server to update one or more instructions in the set of user instructions and communicate the one or more instructions to the console.

20. The system of claim 17, wherein the console comprises a set of recesses that each, in response to a tracker being magnetically attached to the recess, inductively charges the tracker, the console further comprising a ferromagnetic material that is least one of a permanent magnet or a magnetically conductive surface layer to magnetically couple to the tracker.
